(19) United States
(12) Patent Application Publication
(10) Pub. No.: US 2026/0295238 A1
(43) Pub. Date: Oct. 1, 2026

(54) Electrode Assemblies for Applying Tumor Treating Fields (TTFields) to a Subject's Body that Incorporate Silver-Doped Glass Micro-Particles for Sanitization

(71) Applicant: Novocure GmbH, Baar (CH)

(72) Inventor: Uri WEINBERG, Haifa (IL)

(73) Assignee: Novocure GmbH, Baar (CH)

(21) Appl. No.: 19/577,656

(22) Filed: Mar. 25, 2026

Related U.S. Application Data
(60) Provisional application No. 63/779,701, filed on Mar. 28, 2025.

(51) Int. Cl. A61N 1/04; A61L 24/00; A61L 24/02; A61N 1/36

(52) U.S. Cl. CPC A61N 1/0496; A61L 24/0031; A61L 24/02; A61N 1/36002

(57) ABSTRACT

Electrode assemblies for applying Tumor Treating Fields (TTFields) or other types of alternating electric fields to a subject's body typically have a layer of conductive adhesive or conductive gel disposed on the front face of the electrode assembly. Incorporating glass micro-particles that contain at least one of silver, zinc, and copper into this layer or onto the front surface of this layer can provide significant sanitization benefits.

### CROSS REFERENCE TO RELATED APPLICATIONS

This Application claims the benefit of U.S. Provisional Application 63/779,701, filled Mar. 28, 2025, which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 KHz. The alternating electric fields are induced by electrode assemblies (also called transducer arrays) positioned on the subject's skin on opposite sides of the subject's body, and an electrical signal is delivered to each of the electrode assemblies by a respective cable. When an AC voltage is applied between opposing electrode assemblies (via these cables), an AC current is coupled through the electrode assemblies and into the subject's body, which induces the TTFields in a target region within the subject's body.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier (BBB) so that, e.g., chemotherapy drugs can reach the brain.

Because the electrode assemblies contact the subject's skin during use, it can be good practice to disinfect them at the end of the manufacturing process. This is typically done by irradiating them or by exposing them to a gaseous disinfectant.

### SUMMARY OF THE INVENTION

One aspect of the invention is directed to a first apparatus for applying an electrical signal to a subject's body. The first apparatus comprises at least one metal pad, a set of one or more layers of material, a layer of conductive adhesive or conductive gel, and at least 100 glass micro-particles. The at least one metal pad has a front face. The set of one or more layers of material is positioned in front of the at least one metal pad, and the set has (a) a rear layer disposed in contact with the front face of the at least one metal pad and (b) a front layer having a front face. The layer of conductive adhesive or conductive gel is disposed on the front face of the front layer of the set, and the layer of conductive adhesive or conductive gel has a front face. The at least 100 glass micro-particles are disposed within the layer of conductive adhesive or conductive gel and/or disposed on the front face of the layer of conductive adhesive or conductive gel, and each of the at least 100 glass micro-particles contains at least one of silver, zinc, and copper.

Some embodiments of the first apparatus further comprise a release liner disposed on the front face of the layer of conductive adhesive or conductive gel.

Some embodiments of the first apparatus further comprise a release liner disposed on the front face of the layer of conductive adhesive or conductive gel, a flexible backing positioned behind the at least one metal pad, and a sealed pouch. The flexible backing is configured to support the at least one metal pad. The at least one metal pad, the set of one or more layers of material, the layer of conductive adhesive or conductive gel, the at least 100 glass micro-particles, the release liner, and the flexible backing are all enclosed within the sealed pouch.

Some embodiments of the first apparatus further comprise a release liner disposed on the front face of the layer of conductive adhesive or conductive gel, and a flexible backing positioned behind the at least one metal pad. The flexible backing is configured to support the at least one metal pad.

Optionally, in the embodiments described in the previous paragraph, the set of one or more layers of material includes a layer of graphite. Optionally, in the embodiments described in the previous paragraph, the front layer of the set is a layer of graphite. Optionally, in the embodiments described in the previous paragraph, the front layer of the set is a layer of graphite, and the rear layer of the set comprises a conductive adhesive. Optionally, in the embodiments described in the previous paragraph, the set of one or more layers of material includes a layer of ceramic material having a dielectric constant of at least 1000. Optionally, in the embodiments described in the previous paragraph, the set of one or more layers of material includes a layer of an insulating polymer having a dielectric constant of at least 10. Optionally, in the embodiments described in the previous paragraph, the layer of conductive adhesive or conductive gel is a layer of conductive adhesive. Optionally, in the embodiments described in the previous paragraph, the front layer of the set has an area of at least 25 cm². Optionally, in the embodiments described in the previous paragraph, each of the at least 100 glass micro-particles contains silver that is predominantly in the form of silver ions. Optionally, in the embodiments described in the previous paragraph, the at least 100 glass micro-particles are disposed within the layer of conductive adhesive or conductive gel. Optionally, in the embodiments described in the previous paragraph, the at least 100 glass micro-particles are disposed on the front face of the layer of conductive adhesive or conductive gel. Optionally, in the embodiments described in the previous paragraph, each of the at least 100 glass micro-particles is an amorphous and porous glass micro-particle; each of the at least 100 glass micro-particles has a particle size of less than 50 μm; and the glass micro-particles have an average particle size of 0.5-10 μm.

Another aspect of the invention is directed to a second apparatus for applying an electrical signal to a subject's body. The second apparatus comprises at least one metal pad, a layer of conductive adhesive or conductive gel, and at least 100 glass micro-particles. The at least one metal pad has a front face. The layer of conductive adhesive or conductive gel is disposed on the front face of the at least one metal pad, and the layer of conductive adhesive or conductive gel has a front face. The at least 100 glass micro-particles are disposed within the layer of conductive adhesive or conductive gel and/or disposed on the front face of the layer of conductive adhesive or conductive gel, and each of the at least 100 glass micro-particles contains at least one of silver, zinc, and copper.

Some embodiments of the second apparatus further comprise a release liner disposed on the front face of the layer of conductive adhesive or conductive gel.

Some embodiments of the second apparatus further comprise a release liner disposed on the front face of the layer of conductive adhesive or conductive gel, a flexible backing positioned behind the at least one metal pad, and a sealed pouch. The flexible backing is configured to support the at least one metal pad. And the at least one metal pad, the layer of conductive adhesive or conductive gel, the at least 100 glass micro-particles, the release liner, and the flexible backing are all enclosed within the sealed pouch.

Some embodiments of the second apparatus further comprise a release liner disposed on the front face of the layer of conductive adhesive or conductive gel, and a flexible backing positioned behind the at least one metal pad, and the flexible backing is configured to support the at least one metal pad.

Optionally, in the embodiments described in the previous paragraph, the layer of conductive adhesive or conductive gel is a layer of conductive adhesive. Optionally, in the embodiments described in the previous paragraph, the at least one metal pad has an area of at least 25 cm². Optionally, in the embodiments described in the previous paragraph, each of the at least 100 glass micro-particles contains silver that is predominantly in the form of silver ions. Optionally, in the embodiments described in the previous paragraph, the at least 100 glass micro-particles are disposed within the layer of conductive adhesive or conductive gel. Optionally, in the embodiments described in the previous paragraph, the at least 100 glass micro-particles are disposed on the front face of the layer of conductive adhesive or conductive gel. Optionally, in the embodiments described in the previous paragraph, each of the at least 100 glass micro-particles is an amorphous and porous glass micro-particle, each of the at least 100 glass micro-particles has a particle size of less than 50 μm, and the glass micro-particles have an average particle size of 0.5-10 μm.

Another aspect of the invention is directed to a third apparatus for applying an electrical signal to a subject's body. The third apparatus comprises a layer of graphite, a layer of conductive adhesive or conductive gel, and at least 100 glass micro-particles. The layer of graphite has a front face, and the layer of graphite has an area of at least 25 cm². The layer of conductive adhesive or conductive gel is disposed on the front face of the layer of graphite, in electrical contact with the layer of graphite, and the layer of conductive adhesive or conductive gel has a front face. The at least 100 glass micro-particles are disposed within the layer of conductive adhesive or conductive gel and/or disposed on the front face of the layer of conductive adhesive or conductive gel, and each of the at least 100 glass micro-particles contains at least one of silver, zinc, and copper.

Some embodiments of the third apparatus further comprise a first release liner disposed on the front face of the layer of conductive adhesive or conductive gel.

Some embodiments of the third apparatus further comprise a first release liner disposed on the front face of the layer of conductive adhesive or conductive gel, a second release liner, a second layer of conductive adhesive, and a sealed pouch. The layer of graphite, the layer of conductive adhesive or conductive gel, the at least 100 glass micro-particles, the second layer of conductive adhesive, the first release liner, and the second release liner are all enclosed within the sealed pouch. The second layer of conductive adhesive is disposed on a rear face of the layer of graphite. And the second release liner is disposed on a rear face of the second layer of conductive adhesive.

Some embodiments of the third apparatus further comprise a first release liner disposed on the front face of the layer of conductive adhesive or conductive gel, and a sealed pouch. The layer of graphite, the layer of conductive adhesive or conductive gel, the at least 100 glass micro-particles, and the first release liner are all enclosed within the sealed pouch.

Optionally, in the embodiments described in the previous paragraph, the layer of conductive adhesive or conductive gel is a layer of conductive adhesive. Optionally, in the embodiments described in the previous paragraph, the layer of graphite comprises graphite foil made from compressed high purity exfoliated mineral graphite, a sheet of pyrolytic graphite, and/or isotropic graphite. Optionally, in the embodiments described in the previous paragraph, each of the at least 100 glass micro-particles contains silver that is predominantly in the form of silver ions. Optionally, in the embodiments described in the previous paragraph, the at least 100 glass micro-particles are disposed within the layer of conductive adhesive or conductive gel. Optionally, in the embodiments described in the previous paragraph, the at least 100 glass micro-particles are disposed on the front face of the layer of conductive adhesive or conductive gel. Optionally, in the embodiments described in the previous paragraph, each of the at least 100 glass micro-particles is an amorphous and porous glass micro-particle, each of the at least 100 glass micro-particles has a particle size of less than 50 μm, and the glass micro-particles have an average particle size of 0.5-10 μm.

Some embodiments of the third apparatus further comprise at least one metal pad, that (a) is positioned behind the layer of graphite and (b) is disposed in electrical contact with the layer of graphite.

Another aspect of the invention is directed to a fourth apparatus for applying an electrical signal to a subject's body. The fourth apparatus comprises a layer of anisotropic material, a layer of conductive adhesive or conductive gel, and at least 100 glass micro-particles. The layer of anisotropic material has a front face, and a thermal conductivity of the layer of anisotropic material in directions that are parallel to the front face of the layer of anisotropic material is at least 2 times higher than a thermal conductivity of the layer of anisotropic material in a direction that is perpendicular to the front face of the layer of anisotropic material. The layer of anisotropic material has an area of at least 25 cm². The layer of conductive adhesive or conductive gel is disposed on the front face of the layer of anisotropic material, in electrical contact with the layer of anisotropic material. And the layer of conductive adhesive or conductive gel has a front face. The at least 100 glass micro-particles are disposed within the layer of conductive adhesive or conductive gel and/or disposed on the front face of the layer of conductive adhesive or conductive gel, and each of the at least 100 glass micro-particles contains at least one of silver, zinc, and copper.

Some embodiments of the fourth apparatus further comprise a first release liner disposed on the front face of the layer of conductive adhesive or conductive gel.

Some embodiments of the fourth apparatus further comprise a first release liner disposed on the front face of the layer of conductive adhesive or conductive gel, and a sealed pouch. The layer of anisotropic material, the layer of conductive adhesive or conductive gel, the at least 100 glass micro-particles, and the first release liner are all enclosed within the sealed pouch. And the layer of conductive adhesive or conductive gel is a layer of conductive adhesive.

Some embodiments of the fourth apparatus further comprise a first release liner disposed on the front face of the layer of conductive adhesive or conductive gel, a sealed pouch, a second layer of conductive adhesive, and a second release liner. The layer of anisotropic material, the layer of conductive adhesive or conductive gel, the at least 100 glass micro-particles, the first release liner, the second release liner, and the second layer of conductive adhesive are all enclosed within the sealed pouch. In these embodiments, the layer of conductive adhesive or conductive gel is a layer of conductive adhesive; the second layer of conductive adhesive is disposed on a rear face of the layer of graphite; and the second release liner is disposed on a rear face of the second layer of conductive adhesive.

Some embodiments of the fourth apparatus further comprise a first release liner disposed on the front face of the layer of conductive adhesive or conductive gel, and a sealed pouch. The layer of anisotropic material, the layer of conductive adhesive or conductive gel, the at least 100 glass micro-particles, and the first release liner are all enclosed within the sealed pouch.

Optionally, in the embodiments described in the previous paragraph, each of the at least 100 glass micro-particles contains silver that is predominantly in the form of silver ions. Optionally, in the embodiments described in the previous paragraph, the at least 100 glass micro-particles are disposed within the layer of conductive adhesive or conductive gel. Optionally, in the embodiments described in the previous paragraph, the at least 100 glass micro-particles are disposed on the front face of the layer of conductive adhesive or conductive gel. Optionally, in the embodiments described in the previous paragraph, each of the at least 100 glass micro-particles is an amorphous and porous glass micro-particle, each of the at least 100 glass micro-particles has a particle size of less than 50 μm, and the glass micro-particles have an average particle size of 0.5-10 μm.

Some embodiments of the fourth apparatus further comprise at least one metal pad that (a) is positioned behind the layer of anisotropic material and (b) is disposed in electrical contact with the layer of anisotropic material.

### BRIEF DESCRIPTION OF THE DRAWINGS

FIGS. 1A and 1B are plan and section views of one example of an electrode assembly for applying alternating electric fields to a subject's body.

FIG. 1C depicts a variant of the FIGS. 1A-B embodiment.

FIG. 2 depicts one example of how to use the FIGS. 1A-B electrode assembly.

FIGS. 3A and 3B are plan and section views of another example of an electrode assembly for applying alternating electric fields to a subject's body.

FIGS. 4A and 4B are plan and section views of another example of an electrode assembly for applying alternating electric fields to a subject's body.

FIGS. 5A and 5B are plan and section views of another example of an electrode assembly for applying alternating electric fields to a subject's body.

FIGS. 6A and 6B are plan and section views of another electrode assembly for applying alternating electric fields to a subject's body that is divided into two discrete subassemblies that are removably connectable to each other.

FIG. 6C depicts the FIGS. 6A-B electrode assembly after its two subassemblies have been connected to each other.

FIG. 7 depicts one example of how to use the FIGS. 6A-C electrode assemblies to apply alternating electric fields to a target region in a subject's body.

Various embodiments are described in detail below with reference to the accompanying drawings, wherein like reference numerals represent like elements.

### DESCRIPTION OF THE PREFERRED EMBODIMENTS

This application describes alternative approaches for disinfecting electrode assemblies that do not rely on irradiation or exposure to gaseous disinfectants.

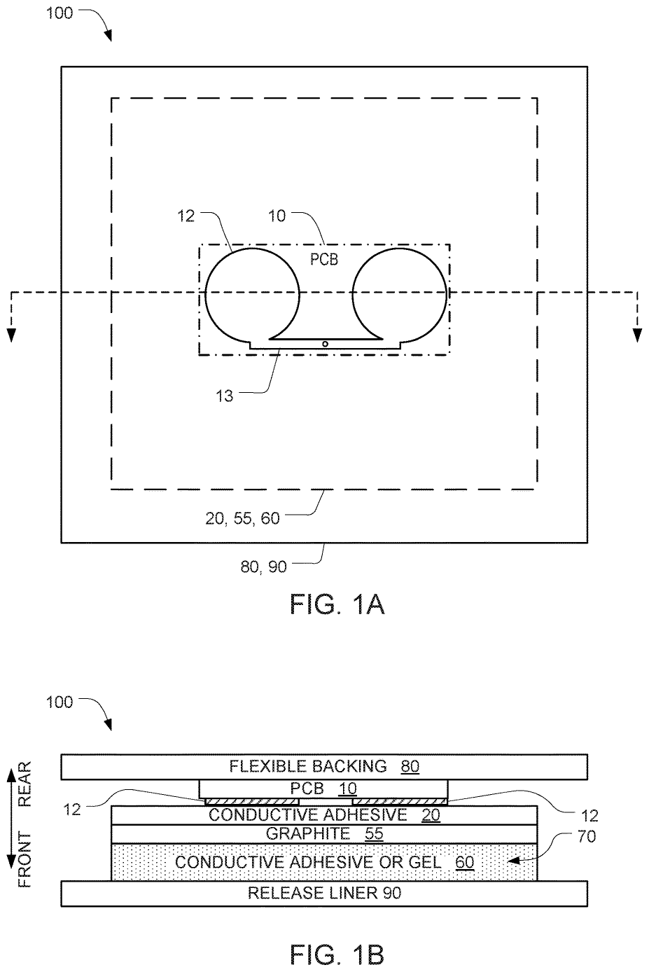

FIGS. 1A and 1B are plan and section views of one example of an electrode assembly 100 for applying alternating electric fields (e.g., TTFields) to a subject's body. In this and all other embodiments described herein, the front of the electrode assembly faces the subject's skin. (See, e.g., the legend on the left side of FIG. 1B.) The electrode assembly 100 includes a PCB 10 with at least one metal pad 12 disposed on the front of the PCB. Note that as used herein, the term “PCB” refers to a printed circuit board, which includes both rigid PCBs (e.g., with copper traces on a rigid epoxy board) and flex circuits (e.g., with copper traces on a flexible polyimide substrate). In the example depicted in FIGS. 1A-B, the metal pads 12 are metal (e.g. copper) pads that are disposed on the front of the PCB 10. When more than one metal pad 12 is included (as depicted in FIGS. 1A-B), all the metal pads 12 can be connected by metal traces 13. Each of the metal pads 12 has a front face.

A set of one or more layers of material is positioned in front of the at least one metal pad. This set includes (a) a rear layer disposed in contact with the front face of the at least one metal pad and (b) a front layer having a front face. In the FIG. 1B embodiment, this set of one or more layers corresponds to (a) a first layer of conductive adhesive 20 disposed on, and positioned in front of, the metal pads 12; and (b) a layer of graphite 55 disposed on, and positioned in front of, the first layer of conductive adhesive 20. The first layer of conductive adhesive 20 conductively couples each of the metal pads 12 to the layer of graphite 55.

Examples of suitable materials for the first layer of conductive adhesive 20 include, but are not limited to, the OMNI-WAVE™ adhesive compositions manufactured and sold by FLEXcon® (Spencer, MA, USA), such as the developmental product FLX068983—FLEXcon® OMNI-WAVE™ TT 200 BLACK H-502 150 POLY H-9 44PP-8; and the adhesives from ADHESIVE RESEARCH, such as ARcare® 8006 electrically conductive adhesive composition manufactured and sold by Adhesives Research, Inc. (Glen Rock, PA, USA). Alternatively, Electrically Conductive Adhesive Transfer Tape 9712 or Electrically Conductive Adhesive Transfer Tape 9713 (both manufactured by 3M, Saint Paul, MN, USA) may also be used.

The layer of graphite 55 (which, in the FIG. 1B embodiment, is the front layer within the set of layers of material that are sandwiched between the metal pads 12 and the layer of conductive adhesive or conductive gel 60) can have an area of at least 10 cm², at least 15 cm², at least 20 cm², at least 25 cm², at least 30 cm², at least 40 cm², or at least 50 cm². Notably, the layer of graphite 55 is both highly thermally conductive and highly electrically conductive in directions that are parallel to the front face of the layer of graphite. It therefore acts to spread both the flow of current and heat in all four directions (i.e., to the right, to the left, into the page, and out of the page in FIG. 1B, which corresponds to right, left, up, and down in FIG. 1A).

Examples of suitable materials for the layer of graphite 55 include, but are not limited to, synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied by MinGraph® 2010A Flexible Graphite, available from Mineral Seal Corp., Tucson, Arizona, USA), graphitized polymer film, e.g., graphitized polyimide film, (including, but not limited to, that supplied by Kaneka Corp., Moka, Tochigi, Japan), and isotropic graphite (including but not limited to isotropic graphite grade G330 available from Tokai Carbon Europe, Oldbury, UK).

In alternative embodiments, a layer of anisotropic material (LoAM) can be used instead of the depicted layer of graphite 55. In these embodiments, the thermal conductivity of the LoAM in directions that are parallel to the front face of the LoAM is at least 2 (or at least 5 or at least 10) times higher than the thermal conductivity of the LoAM in a direction that is perpendicular to the front face of the LoAM.

A layer of conductive adhesive or gel, 60 is disposed on, and positioned in front of, the front face of the front layer of the set of layers of material (which, in the FIG. 1B embodiment, is the front face of the layer of graphite 55). The layer of conductive adhesive or gel 60 is configured to adhere to skin. The layer of conductive adhesive or gel 60 can be any suitable biocompatible adhesive that is designed to be removably affixed to a person's skin including, but not limited to, the same materials described above for the first layer of conductive adhesive 20. Alternatively, the layer of conductive adhesive or gel 60 can be a biocompatible conductive hydrogel.

At least 100 glass micro-particles 70 are disposed within the layer of conductive adhesive or conductive gel 60 and/or disposed on the front face of the layer of conductive adhesive or conductive gel 60. In the embodiment depicted in FIG. 1B, the glass micro-particles 70 are disposed within the layer of conductive adhesive or conductive gel 60 (and are represented schematically by small dots). Alternatively and/or additionally, the glass micro-particles 70 can be disposed on the front face of the layer of conductive adhesive or conductive gel 60, as depicted in FIG. 1C (which is a zoomed-in view of the front right corner of the layer of conductive adhesive or conductive gel 60, in which the glass micro-particles 70 are represented schematically by small circles). Note that the total number of glass micro-particles 70 can vary, and can be, for example, at least 100, at least 500, at least 1000, at least 5000, or at least 10,000).

Importantly, each of the at least 100 glass micro-particles 70 contains at least one of silver, zinc, and copper, and this advantageously provides an antimicrobial effect. In some embodiments, the glass micro-particles 70 contain silver that is predominantly in the form of silver ions, and these embodiments can have a particularly strong antimicrobial effect. In some embodiments, each of the at least 100 glass micro-particles 70 is an amorphous and porous glass micro-particle, each of the at least 100 glass micro-particles 70 has a particle size of less than 50 μm, and the at least 100 glass micro-particles 70 have an average particle size of 0.5-10 μm.

Examples of commercially available products that are suitable for use as the glass micro-particles 70 include TROVO®guard B-K2-04 02 01, TROVO®guard B-K3-04 03 09/14, TROVO®guard B-K3-04 03 05, and TROVO®powder Zn, all of which are available from TROVOtech GmbH (Chemiepark Bitterfeld-Wolfen, Areal A, Edisonstraße 3 06766 Bitterfeld-Wolfen, DE) and/or TROVOcare GmbH & Co. KG (Basler Straße 115 79115 Freiburg DE).

Returning to FIGS. 1A-B, a flexible backing 80 is positioned behind the PCB 10, and this flexible backing 80 is configured to support all of the components 10-60 of the electrode assembly 100 that sit in front of the flexible backing 80. The flexible backing 80 can be made from materials similar to those used in conventional bandages including but not limited to flexible fabric and flexible foam. Optionally, portions of the flexible backing 80 can extend laterally beyond the layers 10-60 (which sit in front of the flexible backing 80) and have a self-adhesive front face configured to help hold the electrode assembly 100 to the subject's skin.

A release liner 90 is disposed on the front face of the layer of conductive adhesive or gel 60. Prior to use, the release liner 90 is peeled away from the layer of conductive adhesive or gel 60, after which the front of the electrode assembly 100 is pressed against the subject's skin. The adhesive nature of the layer of conductive adhesive or gel 60 holds (or at least helps to hold) the electrode assemblies 100 against the subject's skin. The purpose of the release liner 90 is to protect the layer of conductive adhesive or gel 60 from foreign matter that could prevent the layer of conductive adhesive or gel 60 from sticking to the subject's skin.

In some preferred embodiments, the electrode assembly 100 is enclosed within a sealed pouch after the electrode assembly 100 is manufactured. Thus, in these embodiments, the following items will all be enclosed within the sealed pouch: the PCB 10, the at least one metal pad 12; the layer of conductive adhesive or conductive gel 60; the set of one or more layers of material 20, 55 that sit between the at least one metal pad 12 and the layer of conductive adhesive or conductive gel 60; the at least 100 glass micro-particles 70; the release liner 90; and the flexible backing 80.

Notably, enclosing all these items within the sealed pouch will advantageously allow the antimicrobial action of the glass micro-particles 70 to sanitize the electrode assembly 100 during the days or weeks that pass between the time that the items are placed into the pouch and the time that the electrode assembly 100 is eventually removed from the pouch and used. And advantageously, the antimicrobial action of the glass micro-particles can eliminate the need for disinfection by radiation or by exposure to gaseous disinfectants. Furthermore, the antimicrobial action of the glass micro-particles 70 that are disposed on or in the layer of conductive adhesive or conductive gel 60 can also provide an antimicrobial effect after the electrode assembly 100 is positioned on the subject's body, which can ameliorate or minimize adverse reactions on the subject's skin beneath the electrode assemblies 100.

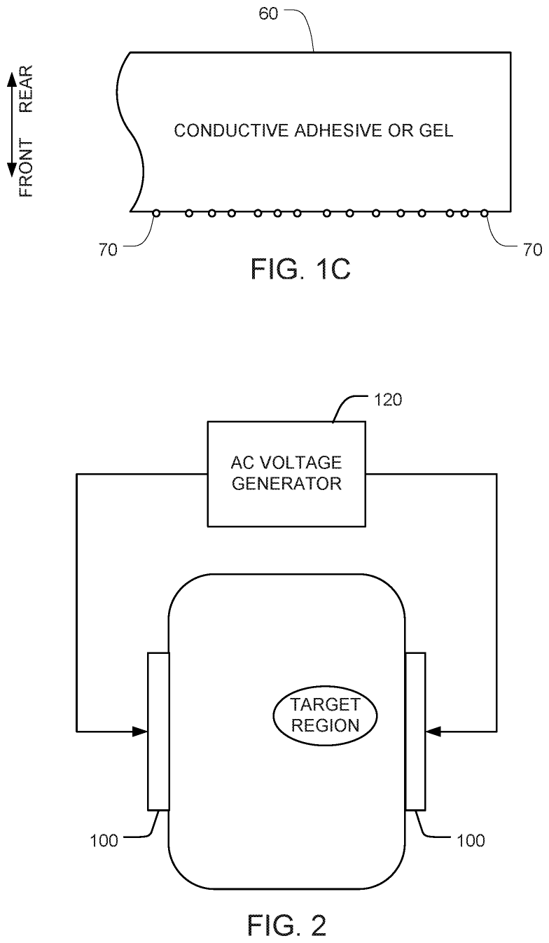

FIG. 2 depicts one example of how to use the electrode assembly 100 depicted in FIGS. 1A-B to apply alternating electric fields (e.g., TTFields) to a target region in a subject's body. This example assumes that we begin with two copies of the electrode assembly 100 depicted in FIGS. 1A-B, that the electrode assemblies 100 have been removed from the pouch, that the release liner 90 has been peeled away from the front face of each of the electrode assemblies 100, and that the front face of the conductive adhesive or gel 60 has been pressed against the subject's skin, as depicted schematically in FIG. 2. The adhesive nature of the layer of conductive adhesive or gel 60 (and optionally the self-adhesive nature of peripheral portions of the flexible backing 80) will hold the electrode assemblies 100 against the subject's skin.

To impose alternating electric fields in the target region, the AC voltage generator 120 applies an AC voltage (e.g., between 50 kHz and 5 MHz, or more commonly 75-300 kHz, 100-250 kHz, or 150-250 kHz) between the metal pads 12 (FIG. 1B) of the first electrode assembly 100 and the metal pads 12 in the second electrode assembly 100. The AC signal from the AC voltage generator 120 arrives at each of the first and second electrode assemblies 100 via a set of cables that route the AC signals (e.g., via the metal traces 13 and other intervening components, not shown) to the metal pads 12 of each of the electrode assemblies 100.

The AC signal that is applied to the metal pads 12 of opposing electrode assemblies 100 will be conductively coupled through all of the conductive layers in front of the layer of metal pads 12 (i.e., the first layer of conductive adhesive 20, the layer of graphite 55, and the layer of conductive adhesive or gel 60. And because the layer of conductive adhesive or gel 60 of each of the opposing electrode assemblies 100 is in contact with the subject's skin on opposite sides of the target region, alternating electric fields will be induced through the target region.

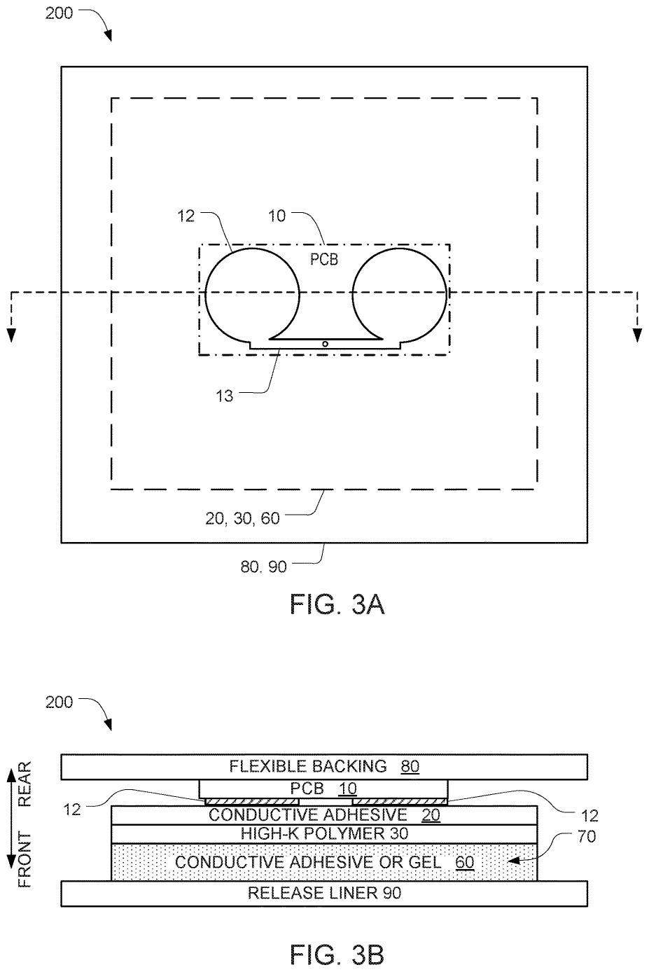

FIGS. 3A and 3B are plan and section views of another example of an electrode assembly 200 for applying alternating electric fields (e.g., TTFields) to a subject's body. The electrode assembly 200 includes a PCB 10 with at least one metal pad 12 disposed on the front of the PCB, as described above in connection with FIGS. 1A-B.

A set of one or more layers of material is positioned in front of the at least one metal pad. This set includes (a) a rear layer disposed in contact with the front face of the at least one metal pad and (b) a front layer having a front face. In the FIG. 3B embodiment, this set of one or more layers corresponds to (a) a first layer of conductive adhesive 20 disposed on, and positioned in front of, the metal pads 12; and (b) an insulating polymer layer 30 with a dielectric constant K of at least 10 disposed on, and positioned in front of, the first layer of conductive adhesive 20. The first layer of conductive adhesive 20 conductively couples each of the metal pads 12 to the insulating polymer layer 30.

Examples of suitable materials for the first layer of conductive adhesive 20 are the same as those described above in connection with FIGS. 1A-B.

The insulating polymer layer 30 (which, in the FIG. 3B embodiment, is the front layer within the set of layers of material that are sandwiched between the metal pads 12 and the layer of conductive adhesive or conductive gel 60) can have an area of at least 10 cm², at least 15 cm², at least 20 cm², at least 25 cm², at least 30 cm², at least 40 cm², or at least 50 cm 2 . The purpose of the insulating polymer layer 30 is to capacitively couple the electrical signal at the metal pads 12 into the subject's body. Examples of suitable materials for the insulating polymer layer 30 include but are not limited to, at least one of Poly(VDF-TrFE-CTFE), Poly(VDF-TrFE-CFE), and Poly(VDF-TrFE-CFE-CTFE), and/or ceramic nanoparticles mixed into at least one of Poly(VDF-TrFE), P(VDF-HFP), PVDF.

A layer of conductive adhesive or gel 60 is disposed on, and positioned in front of, the front face of the front layer of the set of layers of material (which, in the FIG. 3B embodiment, is the front face of the insulating polymer layer 30). Particulars of the layer of conductive adhesive or gel 60 are as described above in connection with FIGS. 1A-B.

At least 100 glass micro-particles 70 are disposed within the layer of conductive adhesive or conductive gel 60 and/or disposed on the front face of the layer of conductive adhesive or conductive gel 60, as described above in connection with FIGS. 1A-C. And the particulars of the glass micro-particles 70 themselves are as described above in connection with FIGS. 1A-C.

Returning to FIGS. 3A-B, a flexible backing 80 is positioned behind the PCB 10, and this flexible backing 80 is configured to support all of the components 10-60 of the electrode assembly 200 that sit in front of the flexible backing 80. Particulars of this flexible backing 80 are as described above in connection with FIGS. 1A-B.

A release liner 90 is disposed on the front face of the layer of conductive adhesive or gel 60. Prior to use, the release liner 90 is peeled away from the layer of conductive adhesive or gel 60, after which the front of the electrode assembly 200 is pressed against the subject's skin, as described above in connection with FIGS. 1A-B.

In some preferred embodiments, the electrode assembly 200 is enclosed within a sealed pouch after the electrode assembly 200 is manufactured. Thus, in these embodiments, the following items will all be enclosed within the sealed pouch: the PCB 10, the at least one metal pad 12; the layer of conductive adhesive or conductive gel 60; the set of one or more layers of material 20, 30 that sit between the at least one metal pad 12 and the layer of conductive adhesive or conductive gel 60; the at least 100 glass micro-particles 70; the release liner 90; and the flexible backing 80. Notably, enclosing all these items within the sealed pouch will provide the same advantages described above in connection with FIGS. 1A-B. Furthermore, the antimicrobial action of the glass micro-particles 70 that are disposed on or in the layer of conductive adhesive or conductive gel 60 can also provide the same advantages described above in connection with FIGS. 1A-B.

Usage of the electrode assemblies 200 is similar to the usage of the electrode assemblies 100 described above in connection with FIG. 2, except that the AC signal that is applied to the metal pads 12 of opposing electrode assemblies 200 will initially be conductively coupled through all of the conductive layers in front of the layer of metal pads 12 (i.e., the first layer of conductive adhesive 20), and will then be capacitively coupled across the insulating polymer layer 30 and into the layer of conductive adhesive or gel 60. And because the layer of conductive adhesive or gel 60 of each of the opposing electrode assemblies 200 is in contact with the subject's skin on opposite sides of the target region, alternating electric fields will be induced through the target region.

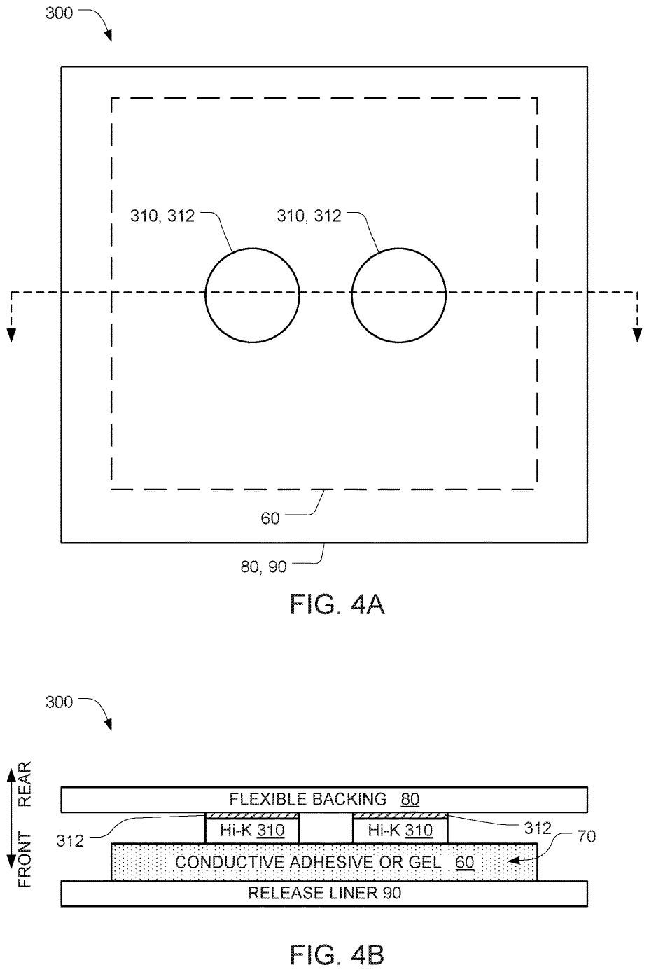

FIGS. 4A and 4B are plan and section views of another example of an electrode assembly 300 for applying alternating electric fields (e.g., TTFields) to a subject's body. The electrode assembly 300 includes a plurality of ceramic plates 310, with a metal pad 312 disposed on the rear face of each of the ceramic plates 310. Each of the ceramic plates is an insulator with a dielectric constant of at least 1000. And each of the metal pads 312 can be formed e.g., by sputtering a metallization layer onto the rear face of a respective ceramic plate 310.

A set of one or more layers of material is positioned in front of the at least one metal pad 312. In this FIGS. 4A-B embodiment, this set includes only a single layer (i.e., the ceramic plate 310), which simultaneously serves as both (a) a rear layer disposed in contact with the front face of the at least one metal pad 312 and (b) a front layer having a front face.

The ceramic plates 310 collectively have an area of at least 10 cm², at least 15 cm², at least 20 cm², at least 25 cm², at least 30 cm², at least 40 cm², or at least 50 cm². The purpose of the ceramic plates 310 is to capacitively couple the electrical signal at the metal pads 312 into the subject's body.

A layer of conductive adhesive or gel, 60 is disposed on, and positioned in front of, the front face of the front layer of the set of layers of material (which, in the FIG. 4B embodiment, is the front face of the ceramic plates 310). Particulars of the layer of conductive adhesive or gel 60 are as described above in connection with FIGS. 1A-B.

At least 100 glass micro-particles 70 are disposed within the layer of conductive adhesive or conductive gel 60 and/or disposed on the front face of the layer of conductive adhesive or conductive gel 60, as described above in connection with FIGS. 1A-C. And the particulars of the glass micro-particles 70 themselves are as described above in connection with FIGS. 1A-C.

Returning to FIGS. 4A-B, a flexible backing 80 is configured to support all of the components 310, 312, and 60 of the electrode assembly 300 that sit in front of the flexible backing 80. Particulars of this flexible backing 80 are as described above in connection with FIGS. 1A-B.

A release liner 90 is disposed on the front face of the layer of conductive adhesive or gel 60. Prior to use, the release liner 90 is peeled away from the layer of conductive adhesive or gel 60, after which the front of the electrode assembly 300 is pressed against the subject's skin, as described above in connection with FIGS. 1A-B.

In some preferred embodiments, the electrode assembly 300 is enclosed within a sealed pouch after the electrode assembly 300 is manufactured. Thus, in these embodiments, the following items will all be enclosed within the sealed pouch: the at least one metal pad 312; the layer of conductive adhesive or conductive gel 60; the set of one or more layers of material (i.e., the ceramic plates 310) that sit between the at least one metal pad 312 and the layer of conductive adhesive or conductive gel 60; the at least 100 glass micro-particles 70; the release liner 90; and the flexible backing 80. Notably, enclosing all these items within the sealed pouch will provide the same advantages described above in connection with FIGS. 1A-B. Furthermore, the antimicrobial action of the glass micro-particles 70 that are disposed on or in the layer of conductive adhesive or conductive gel 60 can also provide the same advantages described above in connection with FIGS. 1A-B.

Usage of the electrode assemblies 300 is similar to the usage of the electrode assemblies 100 described above in connection with FIG. 2, except that the AC signal that is applied to the metal pads 312 of opposing electrode assemblies 300 will be capacitively coupled across the ceramic plates 310 and into the layer of conductive adhesive or gel 60. And because the layer of conductive adhesive or gel 60 of each of the opposing electrode assemblies 300 is in contact with the subject's skin on opposite sides of the target region, alternating electric fields will be induced through the target region.

The embodiments described above in connection with FIGS. 1-4 all include a set of one or more layers of material positioned in front of the at least one metal pad 12/312 and behind the layer of conductive adhesive or conductive gel 60. But in alternative embodiments, the layer of conductive adhesive or conductive gel 60 can be disposed in direct contact with the metal pad(s), as described immediately below.

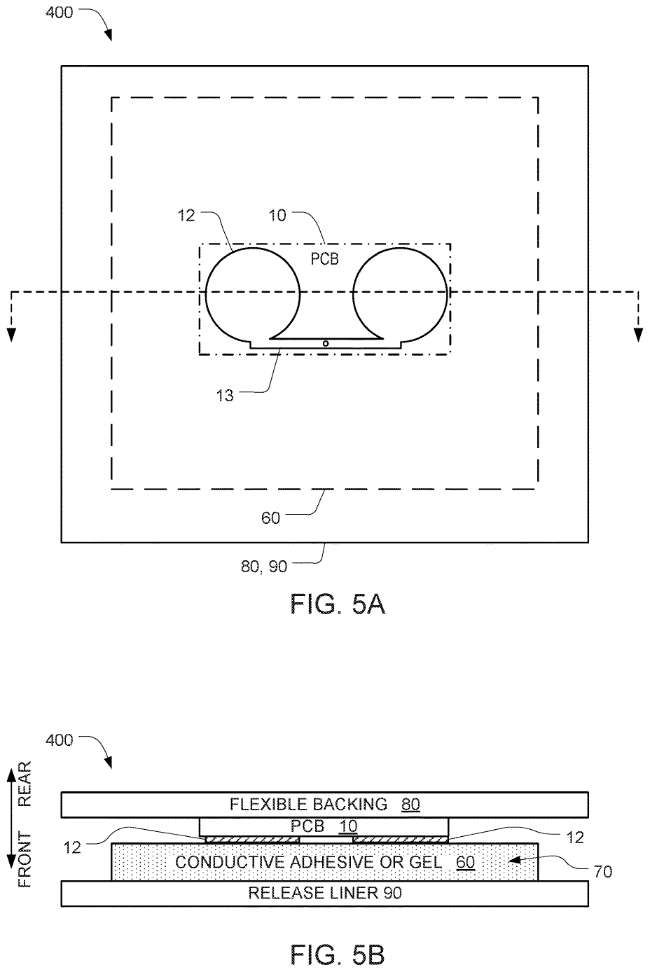

FIGS. 5A and 5B are plan and section views of another example of an electrode assembly 400 for applying alternating electric fields (e.g., TTFields) to a subject's body. The electrode assembly 400 includes a PCB 10 with at least one metal pad 12 disposed on the front of the PCB, e.g., as described above in connection with FIGS. 1A-B.

A layer of conductive adhesive or gel 60 is disposed on, and positioned in front of, the front face of the metal pads 12. Particulars of the layer of conductive adhesive or gel 60 are as described above in connection with FIGS. 1A-B.

At least 100 glass micro-particles 70 are disposed within the layer of conductive adhesive or conductive gel 60 and/or disposed on the front face of the layer of conductive adhesive or conductive gel 60, as described above in connection with FIGS. 1A-C. And the particulars of the glass micro-particles 70 themselves are as described above in connection with FIGS. 1A-C.

Returning to FIGS. 5A-B, a flexible backing 80 is positioned behind the PCB 10, and this flexible backing 80 is configured to support all of the components 10, 12, and 60 of the electrode assembly 400 that sit in front of the flexible backing 80. Particulars of this flexible backing 80 are as described above in connection with FIGS. 1A-B.

A release liner 90 is disposed on the front face of the layer of conductive adhesive or gel 60. Prior to use, the release liner 90 is peeled away from the layer of conductive adhesive or gel 60, after which the front of the electrode assembly 400 is pressed against the subject's skin, as described above in connection with FIGS. 1A-B.

In some preferred embodiments, the electrode assembly 400 is enclosed within a sealed pouch after the electrode assembly 400 is manufactured. Thus, in these embodiments, the following items will all be enclosed within the sealed pouch: the PCB 10, the at least one metal pad 12; the layer of conductive adhesive or conductive gel 60; the at least 100 glass micro-particles 70; the release liner 90; and the flexible backing 80. Notably, enclosing all these items within the sealed pouch will provide the same advantages described above in connection with FIGS. 1A-B. Furthermore, the antimicrobial action of the glass micro-particles 70 that are disposed on or in the layer of conductive adhesive or conductive gel 60 can also provide the same advantages described above in connection with FIGS. 1A-B.

Usage of the electrode assemblies 400 is similar to the usage of the electrode assemblies 100 described above in connection with FIG. 2, except that the AC signal that is applied to the metal pads 12 is conductively coupled directly into the layer of conductive adhesive or gel 60, and does not pass through any intervening layers. And because the layer of conductive adhesive or gel 60 of each of the opposing electrode assemblies 400 is in contact with the subject's skin on opposite sides of the target region, alternating electric fields will be induced through the target region.

The electrode assemblies 100/200/300/400 described above in connection with FIGS. 1-5 each include a plurality of parts that are all incorporated into a single integrated unit. But in alternative embodiments, electric fields can be applied to a subject's body using electrode assemblies that are each divided into two discrete subassemblies that are removably connectable to each other, as described immediately below.

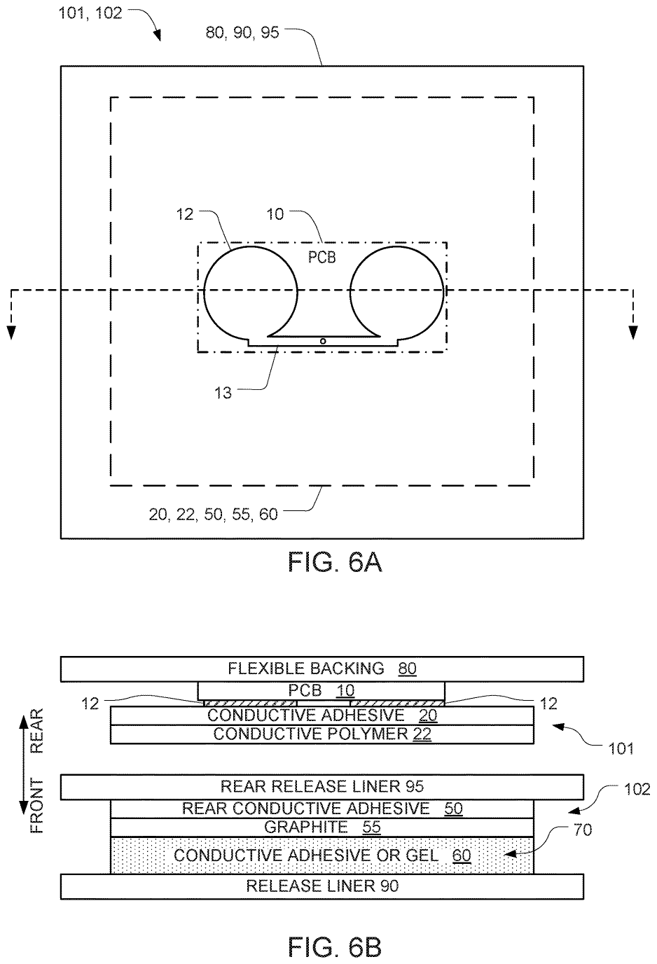

FIGS. 6A and 6B are plan and section views of a two-part electrode assembly for applying alternating electric fields (e.g., TTFields) to a subject's body that is divided into a first (i.e., rear) subassembly 101 and a second (i.e., front) subassembly 102. The two subassemblies 101, 102 can be provided to the end user in a single package or in separate packages. During use, the rear release liner 95 will be removed, and the first and second subassemblies 101, 102 will be pressed against each other and will adhere to each other, as shown in FIG. 6C. The front release liner 90 will also be removed, and the front surface of the second subassembly 102 (i.e., the front surface of the conductive adhesive or gel 60) will be pressed against the subject's skin, as shown in FIG. 6C.

The first subassembly 101 includes a PCB 10 with at least one metal pad 12 disposed on the front of the PCB. Particulars of the PCB 10 and the at least one metal pad 12 are as described above in connection with FIGS. 1A-B.

A first layer of conductive adhesive 20 is disposed on, and positioned in front of, the metal pads 12; and a layer of conductive polymer 22 is disposed on, and positioned in front of, the first layer of conductive adhesive 20. The first layer of conductive adhesive 20 conductively couples each of the metal pads 12 to the layer of conductive polymer 22.

Examples of suitable materials for the first layer of conductive adhesive 20 include the materials described above in connection with FIGS. 1A-B.

The layer of conductive polymer 22 is positioned at the front of the first subassembly 101, so that a front surface of the layer of conductive polymer 22 serves as a front surface of the first subassembly 101. The layer of conductive polymer 22 has an area of at least 10 cm². In some embodiments, this area is at least 15, at least 20, at least 25, at least 30, at least 40, or at least 50 cm².

Examples of suitable materials for the layer of conductive polymer 22 include conductive versions of polymers including, but not limited to silicone, silicone rubber, natural rubber, poly cis-isoprene, polyisobutylene, chloroprene, cis-polybutadiene, styrene-butadiene, styrene-acrylonitrile-butadiene, polyurethane, EPDM, EVA polymers, and perfluoropolymers. Such polymers can be modified to produce conductive versions of the polymers by infusing them with conductive particles, such as, for example, metal particles or carbon particles. Carbon particles may include, for example, carbon flakes, carbon granules, carbon fibers, carbon black powder, graphite powder, carbon nanotubes, carbon nanowires, and the like.

The second subassembly 102 includes a second layer of conductive adhesive 50 positioned at the rear of the second subassembly, so that a rear surface of the second layer of conductive adhesive 50 serves as a rear surface of the second subassembly 102. A layer of graphite 55 is disposed on, and positioned in front of, the second layer of conductive adhesive 50. And a layer of conductive adhesive or gel, 60 is disposed on, and positioned in front of, the layer of graphite 55. The layer of conductive adhesive or gel 60 is configured to adhere to skin.

Examples of suitable materials for the second layer of conductive adhesive 50 include, but are not limited to, the same materials described above for the first layer of conductive adhesive 20. Examples of suitable materials for the layer of graphite 55 include the materials described above in connection with FIGS. 1A-B. The layer of graphite 55 can have an area of at least 10 cm², at least 15 cm², at least 20 cm², at least 25 cm², at least 30 cm², at least 40 cm², or at least 50 cm². And as described above in connection with FIGS. 1A-B, the layer of graphite 55 acts to spread both the flow of current and heat in all four directions (i.e., to the right, to the left, into the page, and out of the page in FIG. 6B, which corresponds to right, left, up, and down in FIG. 6A).

In alternative embodiments, a layer of anisotropic material (LoAM) can be used instead of the depicted layer of graphite 55. In these embodiments, the thermal conductivity of the LoAM in directions that are parallel to the front face of the LoAM is at least 2 (or at least 5 or at least 10) times higher than the thermal conductivity of the LoAM in a direction that is perpendicular to the front face of the LoAM.

Examples of suitable materials for the layer of conductive adhesive or gel 60 include any suitable biocompatible adhesive that is designed to be removably affixed to a person's skin. These may also include, but are not limited to, the same materials described above for the first layer of conductive adhesive 20. Alternatively, the layer of conductive adhesive or gel 60 can be a biocompatible conductive hydrogel.

At least 100 glass micro-particles 70 are disposed within the layer of conductive adhesive or conductive gel 60 and/or disposed on the front face of the layer of conductive adhesive or conductive gel 60, as described above in connection with FIGS. 1A-C. And the particulars of the glass micro-particles 70 themselves are as described above in connection with FIGS. 1A-C.

A flexible backing 80 (e.g., a bandage-like backing) is positioned behind the PCB 10, and this flexible backing 80 is configured to support all of the components 10-22 of the first subassembly 101 that sit in front of the flexible backing 80. In addition, during use (i.e., when the rear release liner 95 has been removed and the first subassembly 101 and the second subassembly 102 have been pressed against each other as shown in FIG. 6C), the flexible backing 80 will also support all of the components 50-60 of the second subassembly 102.

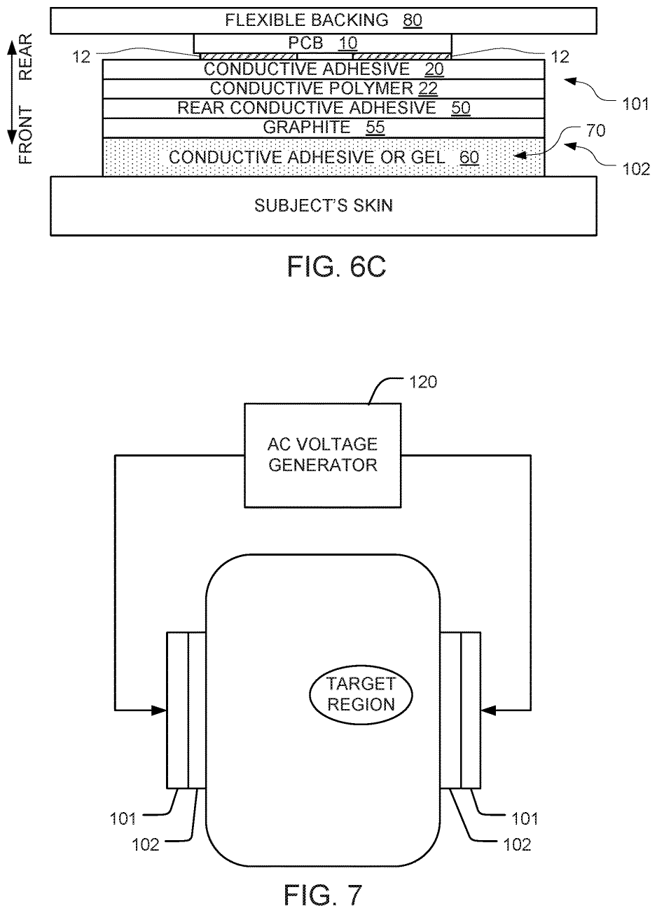

When the first subassembly 101 is positioned in contact with the second subassembly 102 (as seen in FIG. 6C), the front surface of the layer of conductive polymer 22 will be positioned against the rear surface of the second layer of conductive adhesive 50. Due to the adhesive nature of the second layer of conductive adhesive 50, the first and second subassemblies 101, 102 will adhere to each other (until such time when they are pulled apart). In addition,, the contact between the layer of conductive polymer 22 (in the first subassembly 101) and the second layer of conductive adhesive 50 (in the second subassembly 102) allows an AC signal to traverse those two components, as described below in connection with FIG. 7.

In some preferred embodiments, the second subassembly 102 is enclosed within a sealed pouch after the second subassembly 102 is manufactured. Thus, in these embodiments, the following items will all be enclosed within the sealed pouch: the second layer of conductive adhesive 50, the layer of graphite 55, the layer of conductive adhesive or conductive gel 60; the at least 100 glass micro-particles 70; the front release liner 90; and the rear release liner 95. Notably, enclosing all these items within the sealed pouch will provide the same advantages described above in connection with FIGS. 1A-B. Furthermore, the antimicrobial action of the glass micro-particles 70 that are disposed on or in the layer of conductive adhesive or conductive gel 60 can also provide the same advantages described above in connection with FIGS. 1A-B.

FIG. 7 depicts one example of how to use the electrode assemblies 101/102 depicted in FIGS. 6A-C to apply alternating electric fields (e.g., TTFields) to a target region in a subject's body. More specifically, FIG. 7 is a schematic representation of two electrode assemblies 101/102 positioned on a subject's body.

Assuming that the front and rear release liners 90, 95 have been removed, and that the first and second subassemblies 101, 102 have been aligned and pressed together to form an electrode assembly 101/102, one electrode assembly 101/102 is positioned on the subject's skin on one side of the target region, and a second identical electrode assembly 101/102 is positioned on the subject's skin on the opposite side of the target region. Each of the electrode assemblies 101/102 is self adhesive (due to the nature of the layer of conductive adhesive or gel 60, and optionally to peripheral portions of the flexible backing 80), and will therefore stick to the subject's skin.

To impose alternating electric fields in the target region, the AC voltage generator 120 applies an AC voltage (e.g., between 50 kHz and 5 MHz, or more commonly 75-300 kHz, 100-250 kHz, or 150-250 kHz) between the metal pads 12 (FIG. 6C) of the first electrode assembly 101/102 and the metal pads 12 in the second electrode assembly 101/102. The AC signal from the AC voltage generator 120 arrives at each of the first and second electrode assemblies 101/102 via a set of cables that route the AC signals (e.g., via the metal traces 13 and other intervening components, not shown) to the metal pads 12 of the first subassembly 101 of each electrode assembly 101/102.

Because the first and second subassemblies 101, 102 within each electrode assembly are adhered to each other, an AC signal that is applied to the metal pads 12 of opposing electrode assemblies 101/102 will be conductively coupled through all of the conductive layers in front of the layer of metal pads 12 (i.e., the first layer of conductive adhesive 20, the layer of conductive polymer 22, the second layer of conductive adhesive 50, the layer of graphite 55, and the layer of conductive adhesive or gel 60. And because the layer of conductive adhesive or gel 60 of each of the opposing electrode assemblies 101/102 is in contact with the subject's skin on opposite sides of the target region, alternating electric fields will be induced through the target region.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an electrical signal to a subject's body, the apparatus comprising:
at least one metal pad having a front face;
a set of one or more layers of material positioned in front of the at least one metal pad, wherein the set has (a) a rear layer disposed in contact with the front face of the at least one metal pad and (b) a front layer having a front face;
a layer of conductive adhesive or conductive gel disposed on the front face of the front layer of the set, wherein the layer of conductive adhesive or conductive gel has a front face; and
at least 100 glass micro-particles disposed within the layer of conductive adhesive or conductive gel and/or disposed on the front face of the layer of conductive adhesive or conductive gel, wherein each of the at least 100 glass micro-particles contains at least one of silver, zinc, and copper.

2. The apparatus of claim 1, further comprising:
a release liner disposed on the front face of the layer of conductive adhesive or conductive gel; and
a flexible backing positioned behind the at least one metal pad, wherein the flexible backing is configured to support the at least one metal pad.

3. The apparatus of claim 2, further comprising a sealed pouch, wherein the at least one metal pad, the set of one or more layers of material, the layer of conductive adhesive or conductive gel, the at least 100 glass micro-particles, the release liner, and the flexible backing are all enclosed within the sealed pouch.

4. The apparatus of claim 2, wherein the set of one or more layers of material includes a layer of graphite.

5. The apparatus of claim 2, wherein the front layer of the set is a layer of graphite.

6. The apparatus of claim 2, wherein the front layer of the set is a layer of graphite, and the rear layer of the set comprises a conductive adhesive.

7. The apparatus of claim 2, wherein the layer of conductive adhesive or conductive gel is a layer of conductive adhesive.

8. The apparatus of claim 2, wherein the front layer of the set has an area of at least 25 cm².

9. The apparatus of claim 2, wherein each of the at least 100 glass micro-particles contains silver that is predominantly in the form of silver ions.

10. The apparatus of claim 2, wherein the at least 100 glass micro-particles are disposed within the layer of conductive adhesive or conductive gel.

11. The apparatus of claim 2, wherein the at least 100 glass micro-particles are disposed on the front face of the layer of conductive adhesive or conductive gel.

12. The apparatus of claim 2, wherein each of the at least 100 glass micro-particles is an amorphous and porous glass micro-particle, wherein each of the at least 100 glass micro-particles has a particle size of less than 50 μm, and wherein the at least 100 glass micro-particles have an average particle size of 0.5-10 μm.

13. An apparatus for applying an electrical signal to a subject's body, the apparatus comprising:
at least one metal pad having a front face;
a layer of conductive adhesive or conductive gel disposed on the front face of the at least one metal pad, wherein the layer of conductive adhesive or conductive gel has a front face; and
at least 100 glass micro-particles disposed within the layer of conductive adhesive or conductive gel and/or disposed on the front face of the layer of conductive adhesive or conductive gel, wherein each of the at least 100 glass micro-particles contains at least one of silver, zinc, and copper.

14. The apparatus of claim 13, further comprising:
a release liner disposed on the front face of the layer of conductive adhesive or conductive gel; and
a flexible backing positioned behind the at least one metal pad, wherein the flexible backing is configured to support the at least one metal pad.

15. The apparatus of claim 14, further comprising a sealed pouch, wherein the at least one metal pad, the layer of conductive adhesive or conductive gel, the at least 100 glass micro-particles, the release liner, and the flexible backing are all enclosed within the sealed pouch.

16. The apparatus of claim 15, wherein the at least one metal pad has an area of at least 25 cm², wherein each of the at least 100 glass micro-particles is an amorphous and porous glass micro-particle, wherein each of the at least 100 glass micro-particles has a particle size of less than 50 μm, and wherein the at least 100 glass micro-particles have an average particle size of 0.5-10 μm.

17. An apparatus for applying an electrical signal to a subject's body, the apparatus comprising:
a layer of graphite having a front face, wherein the layer of graphite has an area of at least 25 cm²;
a layer of conductive adhesive or conductive gel disposed on the front face of the layer of graphite, in electrical contact with the layer of graphite, wherein the layer of conductive adhesive or conductive gel has a front face; and
at least 100 glass micro-particles disposed within the layer of conductive adhesive or conductive gel and/or disposed on the front face of the layer of conductive adhesive or conductive gel, wherein each of the at least 100 glass micro-particles contains at least one of silver, zinc, and copper.

18. The apparatus of claim 17, further comprising a first release liner disposed on the front face of the layer of conductive adhesive or conductive gel.

19. The apparatus of claim 18, further comprising a sealed pouch, wherein the layer of graphite, the layer of conductive adhesive or conductive gel, the at least 100 glass micro-particles, and the first release liner are all enclosed within the sealed pouch.

20. The apparatus of claim 19, wherein each of the at least 100 glass micro-particles is an amorphous and porous glass micro-particle, wherein each of the at least 100 glass micro-particles has a particle size of less than 50 μm, and wherein the at least 100 glass micro-particles have an average particle size of 0.5-10 μm.
